(19) United States
(12) Patent Application Publication
(10) Pub. No.: US 2022/0069620 A1
(43) Pub. Date: Mar. 3, 2022

(54) RAPID-CHARGING WEARABLE WIRELESS DEVICE

(71) Applicant: Atmosic Technologies Inc., Campbell, CA (US)

(72) Inventors: David Kuochieh Su, Saratoga, CA (US); Masoud Zargari, Saratoga, CA (US)

(21) Appl. No.: 17/367,893

(22) Filed: Jul. 6, 2021

Related U.S. Application Data
(60) Provisional application No. 63/070,172, filed on Aug. 25, 2020.

(51) Int. Cl. H02J 50/00; H02J 50/27; H02J 7/34; H04R 1/10

(52) U.S. Cl. CPC H02J 50/001; H02J 50/27; H02J 7/345; A61B 5/6803; H02J 2207/50; H02J 2310/22; H04R 2201/103; H04R 1/1025

(57) ABSTRACT

A wearable wireless device includes a wireless transceiver coupled to a first antenna, an energy harvester coupled to a second antenna, and a supercapacitor coupled to the energy harvester. The wireless transceiver may be configured to receive a first wireless signal from a paired wireless communication device via the first antenna. The energy harvester may be configured to convert radio-frequency (RF) energy received by the second antenna into a charge. In some instances, the energy harvester may be further configured to charge the supercapacitor in response to a presence of the RF energy. The supercapacitor may be configured to store the charge converted by the energy harvester and provide power to the wireless transceiver. In some instances, the supercapacitor may be further configured to power one or more electronic components of the wearable wireless device with the stored charge.

### CROSS-REFERENCE TO RELATED APPLICATION

This Patent Application claims the benefit of U.S. Provisional Patent Application No. 63/070,172 entitled “RAPID-CHARGING WEARABLE WIRELESS DEVICE” filed on Aug. 25, 2020, which is assigned to the assignee hereof. The disclosures of all prior Applications are considered part of and are incorporated by reference in this Patent Application.

### TECHNICAL FIELD

The present implementations relate generally to wireless devices, and specifically to rapid-charging wearable wireless devices.

### BACKGROUND OF RELATED ART

Wearable wireless devices are often used in conjunction with other wireless devices such as phones, tablets, or laptop computers to enable a user to receive and transmit audio signals or other data without the need for a physical cable. One example of a wearable wireless device may be a wireless headset. Some wireless headsets may consist of two small and lightweight earbuds that may be worn one in each ear. The earbuds can provide the user increased mobility and comfort compared to conventional wired headsets or earphones. Another example of a wearable wireless device may be a fitness tracker to monitor one or more user vital signs including heart rate, body temperature, and the like.

Wearable wireless devices generally include electronic circuits that transmit and receive wireless signals to and from other wireless devices. Additionally, the wearable wireless devices may include one or more batteries to provide power for the electronic circuits. For example, a wearable wireless device may include a battery powered Bluetooth transceiver that transmits and receives Bluetooth audio signals. Typically, the batteries are rechargeable and can power the wireless headset for an extended time period. Recharging batteries may take hours, however, temporarily making the wearable wireless device unavailable for use and negatively affecting the users' experience.

### SUMMARY

The systems, methods and devices of this disclosure each have several innovative aspects, no single one of which is solely responsible for the desirable attributes disclosed herein.

One innovative aspect of the subject matter described in this disclosure can be implemented in a wearable wireless device. In some implementations, the wearable wireless device includes a wireless transceiver coupled to a first antenna of the wearable wireless device, an energy harvester coupled to a second antenna of the wearable wireless device, and a supercapacitor coupled to the energy harvester. The wireless transceiver may be configured to receive a first wireless signal from a paired wireless communication device via the first antenna. The energy harvester may be configured to convert radio-frequency (RF) energy received by the second antenna into a charge. In some instances, the energy harvester may be further configured to charge the supercapacitor in response to a presence of the RF energy. The supercapacitor may be configured to store the charge converted by the energy harvester. In some instances, the supercapacitor may be further configured to power one or more electronic components of the wearable wireless device with the stored charge. In some other instances, the supercapacitor may be further configured to receive a full charge from an external power source within ten seconds.

In some implementations, the wearable wireless device may also include a first audio transducer and a second audio transducer coupled to the wireless transceiver. In some instances, the first audio transducer may be configured to generate a first acoustic signal based on the received first wireless signal. The second audio transducer may be configured receive a second acoustic signal at the second antenna of the wearable wireless device, and the wireless transceiver may be further configured to transmit a second wireless signal based on the received second acoustic signal. In some aspects, the first audio transducer includes a speaker or ear bud, and the second audio transducer includes a microphone.

In other implementations, the wearable wireless device may also include a controller configured to determine an amount of charge stored in the supercapacitor. In some other implementations, the wearable wireless device may include a sensor configured to obtain one or more vital signs of a user. In some aspects, the wireless transceiver may be further configured to transmit a third wireless signal carrying the one or more obtained vital signs to the paired wireless communication device.

Another innovative aspect of the subject matter described in this disclosure can be implemented in a wearable wireless system. In some implementations, the wearable wireless system includes a wearable wireless device and a case. The wearable wireless device may include a wireless transceiver coupled to a first antenna of the wearable wireless device, an energy harvester coupled to a second antenna of the wearable wireless device, and a supercapacitor coupled to the energy harvester. The wireless transceiver may be configured to receive a first wireless signal from a paired wireless communication device via the first antenna. The energy harvester may be configured to convert radio-frequency (RF) energy received by the second antenna into a charge. In some instances, the energy harvester may be further configured to charge the supercapacitor in response to a presence of the RF energy. The supercapacitor may be configured to store the charge converted by the energy harvester. In some instances, the supercapacitor may be further configured to power one or more electronic components of the wearable wireless device with the stored charge. In some other instances, the supercapacitor may be further configured to receive a full charge from an external power source within ten seconds. The case may be configured to store the wearable wireless device while the wearable wireless device is not in use. In some instances, the case may include a battery configured to selectively deliver charge to the supercapacitor

In some implementations, the wearable wireless device may also include a first audio transducer and a second audio transducer coupled to the wireless transceiver. In some instances, the first audio transducer may be configured to generate a first acoustic signal based on the received first wireless signal. The second audio transducer may be configured receive a second acoustic signal at the second antenna of the wearable wireless device, and the wireless transceiver may be further configured to transmit a second wireless signal based on the received second acoustic signal. In some aspects, the first audio transducer includes a speaker or ear bud, and the second audio transducer includes a microphone.

In other implementations, the wearable wireless device may also include a controller configured to determine an amount of charge stored in the supercapacitor. In some other implementations, the wearable wireless device may include a sensor configured to obtain one or more vital signs of a user. In some aspects, the wireless transceiver may be further configured to transmit a third wireless signal carrying the one or more obtained vital signs to the paired wireless communication device.

### BRIEF DESCRIPTION OF THE DRAWINGS

Aspects of the present disclosure are illustrated by way of example and are not intended to be limited by the figures of the accompanying drawings.

FIG. 1 shows a wireless communication system within which aspects of the present disclosure may be implemented.

FIG. 2 is a block diagram of an example wearable wireless system.

FIG. 3 is a block diagram of an example wireless headset.

Like numbers reference like elements throughout the drawings and specification.

### DETAILED DESCRIPTION

In the following description, numerous specific details are set forth such as examples of specific components, circuits, and processes to provide a thorough understanding of the disclosure. The term “coupled” as used herein means coupled directly to or coupled through one or more intervening components or circuits. Also, in the following description and for purposes of explanation, specific nomenclature is set forth to provide a thorough understanding of the example implementations. However, it will be apparent to one skilled in the art that these specific details may not be required to practice the example implementations. In other instances, well-known circuits and devices are shown in block diagram form to avoid obscuring the disclosure. Any of the signals provided over various buses described herein may be time-multiplexed with other signals and provided over one or more common buses. Additionally, the interconnection between circuit elements or software blocks may be shown as buses or as single signal lines. Each of the buses may alternatively be a single signal line, and each of the single signal lines may alternatively be buses, and a single line or bus might represent any one or more of a myriad of physical or logical mechanisms for communication between components. The example implementations are not to be construed as limited to specific examples described herein but rather to include within their scope all implementations defined by the appended claims.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory computer-readable storage medium comprising instructions that, when executed, performs one or more of the methods described below. The non-transitory computer-readable storage medium may form part of a computer program product, which may include packaging materials.

The various illustrative logical blocks, modules, circuits and instructions described in connection with the implementations disclosed herein may be executed by one or more processors, such as one or more digital signal processors (DSPs), general purpose microprocessors, application specific integrated circuits (ASICs), application specific instruction set processors (ASIPs), field programmable gate arrays (FPGAs), or other equivalent integrated or discrete logic circuitry. The term “processor,” as used herein may refer to any of the foregoing structure or any other structure suitable for implementation of the techniques described herein. In addition, in some aspects, the functionality described herein may be provided within dedicated software modules or hardware modules configured as described herein. Also, the techniques could be fully implemented in one or more circuits or logic elements. A general-purpose processor may be a microprocessor, but in the alternative, the processor may be any conventional processor, controller, microcontroller, or state machine. A processor may also be implemented as a combination of computing devices (such as a combination of a DSP and a microprocessor), a plurality of microprocessors, one or more microprocessors in conjunction with a DSP core, or any other suitable configuration.

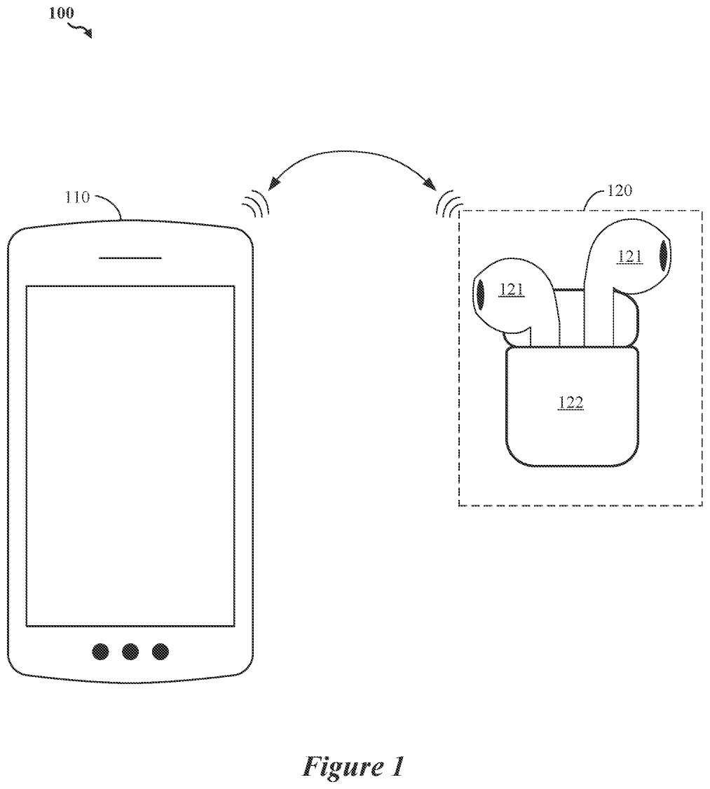

FIG. 1 shows a wireless communication system 100 within which aspects of the present disclosure may be implemented. The wireless communication system 100 may include a wireless device 110 and a wearable wireless system 120. Example wireless devices may include a cell phone, personal digital assistant (PDA), tablet device, laptop computer, or any other suitable portable device. The wireless device 110 may also be referred to as a user equipment (UE), a subscriber station, a mobile unit, a subscriber unit, a wireless unit, a remote unit, a mobile device, a wireless device, a wireless communications device, a remote device, a mobile subscriber station, an access terminal, a mobile terminal, a wireless terminal, a remote terminal, a handset, a user agent, a mobile client, a client, or some other suitable terminology.

The wearable wireless system 120 may include any feasible wearable wireless device 121 and a case 122. For simplicity, the wearable wireless device 121 is depicted as stereo earbuds. In other implementations, the wearable wireless device 121 may include wireless headphones, mono or stereo wireless headsets, wireless fitness monitors or trackers, wireless timekeeping devices, mono or stereo earbuds, or the like. The wearable wireless device 121 may include electronic circuitry (not shown for simplicity) to transmit and/or receive wireless signals, including wireless audio signals, to and/or from the wireless device 110. For example, the wearable wireless device 121 may be implemented as a mono or stereo over-the-ear headset, as one or more earbuds (as shown here) or as any other technically feasible audio reproduction and/or capture device. The wearable wireless device 121 may generate acoustic signals for a user to hear based on the received wireless audio signals. In addition, the wearable wireless device 121 may receive acoustic signals from the user (via a microphone, for example) and transmit wireless audio signals based on the received acoustic signals to the wireless device 110. In another example, the wearable wireless device 121 may be a wearable fitness tracker that may collect heart rate or any other user health information and transmit the user health information to the wireless device 110.

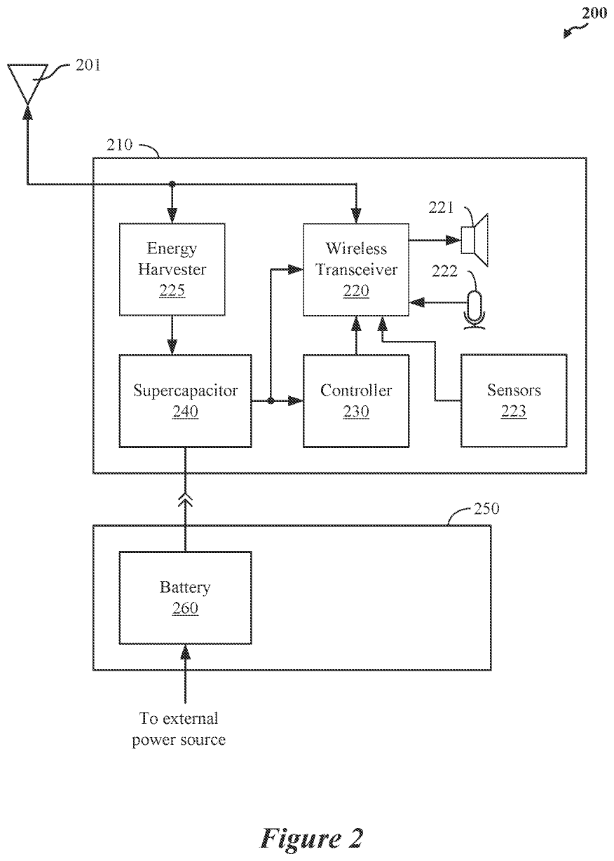

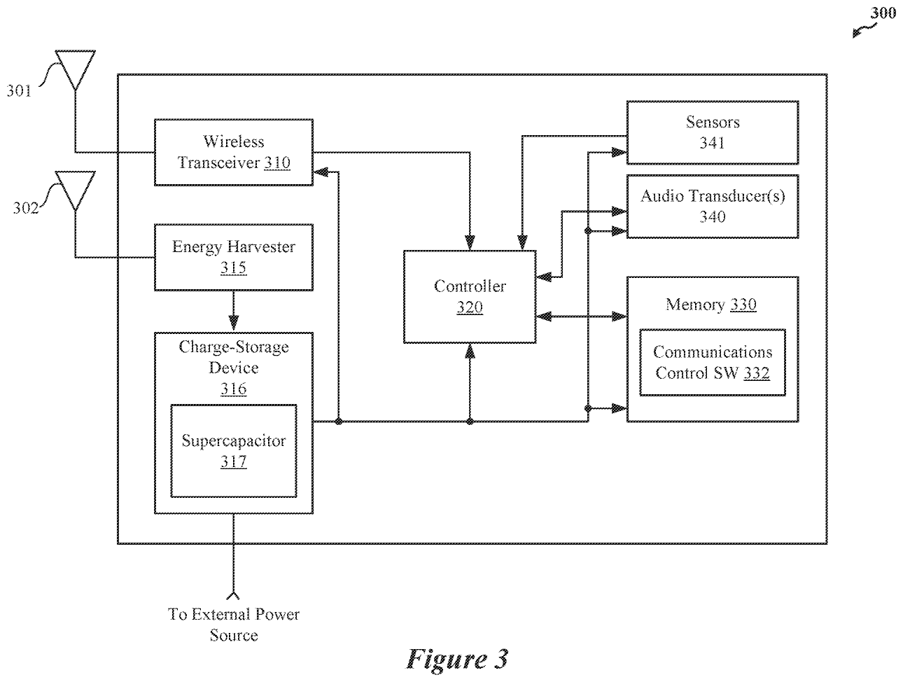

The wearable wireless device 121 may include one or more supercapacitors (not shown for simplicity) to provide power for some or all of the electronic circuitry in the wearable wireless device121. A supercapacitor may be a high-capacity capacitor with a capacitance value much higher than conventional capacitors. For example, a supercapacitor may store 10 to 100 times more energy per unit volume than a conventional capacitor, such as an electrolytic capacitor. In some implementations, the wearable wireless device 121 also may include an energy harvester to harvest energy from radio frequency (RF) signals transmitted by other devices including, but not limited to, the wireless device 110. In some cases, the harvested energy may replenish charge in the supercapacitor. The supercapacitor and energy harvester are described in more detail with respect to FIGS. 2 and 3.

The case 122 may be designed to receive, enclose, and/or store the wearable wireless device 121. In some implementations, the case 122 may include a battery (not shown for simplicity) to provide charge for the one or more supercapacitors included in the wearable wireless device 121. In some implementations, charge from the battery is transferred to the wearable wireless device 121 when the wearable wireless device 121 is inserted, placed into, or becomes proximate to the case 122.

For ease of explanation and clarity, the wireless communication system 100 depicts a single wireless device 110 and a single wearable wireless system 120. In other implementations, the wireless communication system 100 may include any technically feasible number of wireless devices and/or wearable wireless systems. The wireless device 110 and the wearable wireless device 121 may communicate with each other via one or more technically feasible wireless communication protocols. In some implementations, the wireless device 110 and the wearable wireless device 121 may communicate with each other (and with other devices not shown for simplicity) via Wi-Fi, Bluetooth®, Bluetooth Low Energy (BLE), Long Term Evolution (LTE), or any other suitable communication protocol. In some other implementations, the wireless device 110 and wearable wireless device 121 may operate within the 900 MHz band, the 2.4 GHz industrial, scientific, and medical (ISM) band, the 5 GHz ISM band, the 60 GHz band or any other technically feasible frequency band.

FIG. 2 is a block diagram of an example wearable wireless system 200. The wearable wireless system 200 may be an implementation of the wearable wireless system 120 of FIG. 1. The wearable wireless system 200 may include a wearable wireless device 210 and a case 250. The wearable wireless device 210 may be an implementation of the wearable wireless device 121 and the case 250 may be an implementation of the case 122. Although only one wearable wireless device 210 and one case 250 are shown, in other implementations, the wearable wireless system 200 may include any number of wearable wireless devices and cases. For example, the wearable wireless system 200 may include two wearable wireless devices 210 (for example, implemented as earbuds, one for each ear) and one case 250.

The wearable wireless device 210 may include an antenna 201, a wireless transceiver 220, a first audio transducer 221, a second audio transducer 222, sensors 223, an energy harvester 225, a controller 230, and a supercapacitor 240. The wireless transceiver 220 may transmit and/or receive wireless signals, such as wireless audio signals and/or sensor data, through the antenna 201. For example, the wireless transceiver 220 may transmit and receive Wi-Fi, Bluetooth, BLE, and/or LTE wireless signals. In another example, the wireless transceiver 220 may transmit sensor data collected by the sensors 223. Although only one antenna 201 is shown associated with the wearable wireless device 210, in other implementations, the wearable wireless device 210 may include any feasible number of antennas.

The wireless transceiver 220 may be coupled to the first audio transducer 221, the second audio transducer 222, and the sensors 223. The first audio transducer 221 may be an audio reproduction device such as a speaker or earphone. In one implementation, a first wireless audio signal is received via the wireless transceiver 220, converted to a first acoustic signal, and reproduced through the first audio transducer 221. The second audio transducer 222 may be an audio capture device, such as a microphone. Thus, in another implementation, a second acoustic signal may be captured by the second audio transducer 222, converted to a second wireless audio signal, and transmitted via the wireless transceiver 220. The sensors 223 may include capacitance sensors, resistance sensors, optical sensors, pressure sensors, temperature sensors, or any other feasible sensors. In one implementation, the sensors 223 may detect one or more user physical attributes (e.g., vital signs) such as heart rate, body temperature, respiration rate, walking steps, and the like. The associated sensor data may be transmitted to another wireless device (not shown for simplicity) via the wireless transceiver 220

The controller 230 may control, at least in part, the wireless transceiver 220. For example, the controller 230 may direct the wireless transceiver 220 to “pair” with another wireless device or enable the wireless transceiver 220 to join a Wi-Fi network. The controller 230 may also cause the wireless transceiver 220 to transmit and/or receive wireless signals. The supercapacitor 240 may provide power to the wireless transceiver 220 and the controller 230. In some implementations, the supercapacitor 240 may be replaced with any other suitable charge-storage device. In some implementations, the supercapacitor 240 may provide sufficient power for the wireless transceiver 220 and/or the controller 230 to operate for a time period. For example, the supercapacitor 240 may provide sufficient power to operate the wireless transceiver 220 and the controller 230 for two hours. In other implementations, the supercapacitor 240 may provide sufficient power to enable the wireless transceiver 220 and the controller 230 to operate for any other feasible time period.

The energy harvester 225 may be coupled to, and receive RF energy from, the antenna 201. The energy harvester 225 may harvest (convert) the RF energy into power (e.g., a voltage and/or current) for the wearable wireless device 210. In one implementation, the energy harvester 225 may be coupled to the supercapacitor 240. Thus, harvested power from the energy harvester 225 may replenish the charge in the supercapacitor 240 that may have been consumed by the wireless transceiver 220 and/or the controller 230.

The case 250 may include a battery 260. The battery 260 may be a rechargeable battery that can be recharged via an external power source such as an external power supply or the like (not shown for simplicity). The battery 260 may be coupled to the supercapacitor 240 when, for example, the wearable wireless device 210 is placed in or near the case 250. The battery 260 may fully charge the supercapacitor 240 in as quickly as a few seconds (e.g., ten seconds or less). In contrast, recharging a conventional rechargeable battery may take several minutes or hours. The charge time of the supercapacitor 240 may be limited by circuit resistance (which may be low) and peak output current capability of the battery 260 (which may be high). On the other hand, the charge time of a conventional rechargeable battery may be determined by an electro-chemical reaction speed, which may be relatively fixed and lengthy. The comparatively short charge times of the supercapacitor 240 may advantageously reduce the downtime during which the wearable wireless device 210 may be unavailable to the user. In some cases, the charge time of the supercapacitor 240 may appear instantaneous to the user. The energy harvester 225 may further reduce downtime by replenishing charge in the supercapacitor 240 whenever sufficient RF energy is available.

In some implementations, the controller 230 may determine that the supercapacitor 240 is in a low charge state (e.g., the controller 230 determines that the wearable wireless device 210 may deplete the charge of the supercapacitor 240 within a few minutes or any other predetermined time period). After detecting a low charge state, the controller 230 may cause a tone to be emitted by the first audio transducer 221 to alert the user. In other implementations, the controller 230 may perform any other technically feasible operation based on supercapacitor 240 charge levels.

FIG. 3 is a block diagram of an example wearable wireless device 300. The wearable wireless device 300 may be an implementation of the wearable wireless device 121 of FIG. 1 or the wearable wireless device 210 of FIG. 2. The wearable wireless device 300 may include antennas 301 and 302, a wireless transceiver 310, an energy harvester 315, a charge-storage device 316, a controller 320, a memory 330, one or more audio transducers 340, and one or more sensors 341. The wireless transceiver 310 may be an implementation of the wireless transceiver 220 of FIG. 2. The audio transducers 340 may include a speaker, a microphone, or any other technically feasible audio transducer. The sensors 341 may include any feasible sensor.

The wireless transceiver 310 may be coupled to antenna 301 and include circuits, components, and/or devices to enable the wearable wireless device 300 to transmit and/or receive RF communication signals. For example, the wireless transceiver 310 may transmit and receive wireless audio signals via Wi-Fi, Bluetooth, BLE, LTE, or any other technically feasible wireless protocol. In another example, the wireless transceiver 310 may transmit sensor data from the sensors 341.

The charge-storage device 316 may provide power for the wearable wireless device 300. For example, the charge-storage device 316 may provide power to the wireless transceiver 310, the controller 320, the audio transducers 340, the sensors 341, and the memory 330. In some implementations, the charge-storage device 316 may include a supercapacitor 317 to store power (e.g., charge) for the wearable wireless device 300. The charge-storage device 316 may receive charge from an external power source, not shown for simplicity.

The energy harvester 315 may be coupled to, and receive RF energy from, the antenna 302. The energy harvester 315 may harvest (convert) the RF energy into power (e.g., a voltage and/or current) to supply charge to the charge-storage device 316 and/or power, at least partially, the wearable wireless device 300. Although the wearable wireless device 300 shows only one energy harvester 315, in other implementations, the wearable wireless device 300 may include any technically feasible number of energy harvesters.

The wireless transceiver 310 may be coupled to the controller 320. The controller 320 may cause the wireless transceiver 310 to transmit and/or receive wireless communication signals. In addition, the controller 320 may monitor charge levels of the charge-storage device 316.

The memory 330 may include a non-transitory computer-readable storage medium (such as one or more nonvolatile memory elements, such as EPROM, EEPROM, Flash memory, a hard drive, etc.) that may store a communications control software (SW) module 332 to control wireless transmission and reception operations of the wireless transceiver 310. The controller 320, which may be coupled to the wireless transceiver 310, and the memory 330, may be any one or more suitable controllers or processors capable of executing scripts or instructions of one or more software programs stored in the wearable wireless device 300 (e.g., within the memory 330). In some implementations, the controller 320 may be implemented with a hardware controller, a processor, a state machine or other circuits to provide the functionality of the controller 320 executing instructions stored in the memory 330.

The controller 320 may execute the communications control SW module 332 to transmit and/or receive wireless signals via the wireless transceiver 310. In one example, execution of the communications control SW module 332 may cause the wireless transceiver 310 to receive a first wireless audio signal and reproduce an associated acoustic audio signal through one of the audio transducers 340. In another example, execution of the communications control SW module 332 may cause one of the audio transducers 340 to capture an acoustic audio signal and cause the wireless transceiver 310 to transmit an associated second wireless audio signal. In still another example, execution of the communications control SW module 332 may cause the wireless transceiver 310 to transmit sensor data.

As used herein, a phrase referring to “at least one of” or “one or more of” a list of items refers to any combination of those items, including single members. For example, “at least one of: a, b, or c” is intended to cover the possibilities of: a only, b only, c only, a combination of a and b, a combination of a and c, a combination of b and c, and a combination of a and b and c.

The various illustrative components, logic, logical blocks, modules, circuits, operations and algorithm processes described in connection with the implementations disclosed herein may be implemented as electronic hardware, firmware, software, or combinations of hardware, firmware or software, including the structures disclosed in this specification and the structural equivalents thereof. The interchangeability of hardware, firmware and software has been described generally, in terms of functionality, and illustrated in the various illustrative components, blocks, modules, circuits and processes described herein. Whether such functionality is implemented in hardware, firmware or software depends upon the particular application and design constraints imposed on the overall system.

Various modifications to the implementations described in this disclosure may be readily apparent to persons having ordinary skill in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Additionally, various features that are described in this specification in the context of separate implementations also can be implemented in combination in a single implementation. Conversely, various features that are described in the context of a single implementation also can be implemented in multiple implementations separately or in any suitable subcombination. As such, although features may be described herein as acting in particular combinations, and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flowchart or flow diagram. However, other operations that are not depicted can be incorporated in the example processes that are schematically illustrated. For example, one or more additional operations can be performed before, after, simultaneously, or between any of the illustrated operations. In some circumstances, multitasking and parallel processing may be advantageous. Moreover, the separation of various system components in the implementations described herein should not be understood as requiring such separation in all implementations, and it should be understood that the described program components and systems can generally be integrated together in a single software product or packaged into multiple software products.

What is claimed is:

1. A wearable wireless device comprising:
a wireless transceiver coupled to a first antenna of the wearable wireless device, the wireless transceiver configured to receive a first wireless signal from a paired wireless communication device via the first antenna;
an energy harvester coupled to a second antenna of the wearable wireless device, the energy harvester configured to convert radio-frequency (RF) energy received by the second antenna into a charge; and
a supercapacitor coupled to the energy harvester, the supercapacitor configured to store the charge converted by the energy harvester.

2. The wearable wireless device of claim 1, wherein the supercapacitor is further configured to power one or more electronic components of the wearable wireless device with the stored charge.

3. The wearable wireless device of claim 1, wherein the supercapacitor is further configured to receive a full charge from an external power source within ten seconds.

4. The wearable wireless device of claim 1, further comprising:
a first audio transducer coupled to the wireless transceiver, the first audio transducer configured to generate a first acoustic signal based on the received first wireless signal.

5. The wearable wireless device of claim 4, further comprising:
a second audio transducer coupled to the wireless transceiver, the second audio transducer configured receive a second acoustic signal at the second antenna of the wearable wireless device, wherein the wireless transceiver is further configured to transmit a second wireless signal based on the received second acoustic signal.

6. The wearable wireless device of claim 5, wherein the first audio transducer comprises a speaker or ear bud, and the second audio transducer comprises a microphone.

7. The wearable wireless device of claim 1, further comprising a controller configured to determine an amount of charge stored in the supercapacitor.

8. The wearable wireless device of claim 1, further comprising:
a sensor configured to obtain one or more vital signs of a user, wherein the wireless transceiver is further configured to transmit a third wireless signal carrying the one or more obtained vital signs to the paired wireless communication device.

9. The wearable wireless device of claim 1, wherein the energy harvester is further configured to charge the supercapacitor in response to a presence of the RF energy.

10. A wearable wireless system comprising:
a wearable wireless device including:
a wireless transceiver coupled to a first antenna of the wearable wireless device, the wireless transceiver configured to receive a first wireless signal from a paired wireless communication device via the first antenna;
an energy harvester coupled to a second antenna of the wearable wireless device, the energy harvester configured to convert radio-frequency (RF) energy received by the second antenna into a charge; and
a supercapacitor coupled to the energy harvester, the supercapacitor configured to store the charge converted by the energy harvester; and
a case configured to store the wearable wireless device while the wearable wireless device is not in use, the case including a battery configured to selectively deliver charge to the supercapacitor.

11. The wearable wireless system of claim 10, wherein the supercapacitor is further configured to power one or more electronic components of the wearable wireless device with the stored charge.

12. The wearable wireless system of claim 10, wherein the supercapacitor is further configured to receive a full charge from an external power source within ten seconds.

13. The wearable wireless system of claim 10, further comprising:
a first audio transducer coupled to the wireless transceiver, the first audio transducer configured to generate a first acoustic signal based on the received first wireless signal.

14. The wearable wireless system of claim 13, further comprising:
a second audio transducer coupled to the wireless transceiver, the second audio transducer configured receive a second acoustic signal at the second antenna of the wearable wireless device, wherein the wireless transceiver is further configured to transmit a second wireless signal based on the received second acoustic signal.

15. The wearable wireless system of claim 14, wherein the first audio transducer comprises a speaker or ear bud, and the second audio transducer comprises a microphone.

16. The wearable wireless system of claim 10, further comprising a controller configured to determine an amount of charge stored in the supercapacitor.

17. The wearable wireless system of claim 10, further comprising:
a sensor configured to obtain one or more vital signs of a user, wherein the wireless transceiver is further configured to transmit a third wireless signal carrying the one or more obtained vital signs to the paired wireless communication device.

18. The wearable wireless system of claim 10, wherein the energy harvester is further configured to charge the supercapacitor in response to a presence of the RF energy.
